(19) United States
(12) Patent Application Publication
(10) Pub. No.: US 2008/0214479 A1
(43) Pub. Date: Sep. 4, 2008

(54) Methods of mimicking the metabolic effects of caloric restriction by administration of mannoheptulose

(75) Inventors: Josef Pitha, Rockville, MD (US); George Roth, Pylesville, MD (US); Michael G. Hayek, Dayton, OH (US); Stefan Patrick Massimino, Portland, OR (US); Michael A. Ceddia, Newburgh, IN (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignees: The lams Company, Cincinnati, OH (US); GeroTech, Inc., Pylesville, MD (US)

(21) Appl. No.: 12/082,710

(22) Filed: Apr. 14, 2008

Related U.S. Application Data
(63) Continuation of application No. 11/313,198, filed on Dec. 20, 2005.
Application No. 11/313,198 is a continuation of application No. 09/950,052, filed on Sep. 12, 2001, now abandoned.
Application No. 09/950,052 is a continuation-in-part of application No. 08/899,877, filed on Jul. 24, 1997, now abandoned.

(51) Int. Cl. A61K 31/70; A61P 3/08

(52) U.S. Cl. 514/23

(57) ABSTRACT

A method of obtaining beneficial biological results associated with caloric restriction may be gained by administration of a composition containing at least one active agent which blocks metabolism of glucose as a source of energy in cells in glucose metabolism blocking effective amounts to an animal in need thereof.

### CROSS REFERENCE TO RELATED APPLICATION

This is a continuation application of U.S. patent application Ser. No. 11/313,198, filed Dec. 20, 2005, now pending, which is a continuation of U.S. patent application Ser. No. 09/950,052, filed Sep. 12, 2001, now abandoned, which is a continuation-in-part of U.S. patent application Ser. No. 08/889,877, filed Jul. 8, 1997, now abandoned.

### FIELD OF THE INVENTION

This invention relates to the use of glucose anti-metabolites to alter utilization of glucose or other energy sources and to mimic metabolic effects of caloric restriction.

### BACKGROUND OF THE INVENTION

Biological theories correctly predict the finding that a restriction of caloric intake by food deprivation slows down certain undesirable cellular processes in laboratory animals, many associated with aging and age-related diseases.

It is also known that hyperinsulinemia is a risk factor associated with several such disease processes, including heart disease and diabetes (Balkau and Eschwege. Diabetes Obes. Metab. 1 (Suppl 1): S23-31, 1999). The avoidance of hyperinsulinemia should be a goal for treatment of many individuals.

Glucose anti-metabolites such as 2 deoxy-D-glucose are compounds related to glucose. However, due to structural differences from glucose such compounds block or inhibit certain aspects of carbohydrate metabolism (Rezek, et al., J. Nutr. 106:143-157, 1971). These anti-metabolites exert a number of physiological effects, including reduction of body weight, decrease in plasma insulin levels, reduction of body temperature, retardation of tumor formation and growth, and elevation of circulating glucocorticoid hormone concentrations. (For a review see Roth et al., Ann. NY Acad. Sci. 928: 305-315, 2001.) These effects result from inhibition of carbohydrate metabolism. Reduced insulin levels and body temperature are two of the most reliable indicators of this altered metabolic profile (Masoro et al., J. Gerontol. Biol. Sci. 47:B202-B208, 1992; Koizumi et al., J. Nutr. 117: 361-367; 1987; Lane et al., Proc. Nat. Acad. Sci. 93:4154-4164, 1996). Intervention designed to provide beneficial physiological regulation of biological processes while allowing animals to avoid undesirable effects of caloric restriction would provide improved health benefits.

### SUMMARY OF THE INVENTION

It is the purpose of this invention, to provide a means of mimicking the beneficial metabolic effects of caloric restriction by carefully controlled administration of anti-metabolites of glucose. Some preferred antimetabolites for use according to the teachings herein include ketoses (mannoheptulose) and anhydrosugars (anhydroglucitols and anhydromannitols) that are structurally similar to glucose. Using methods of the invention, it is possible to obtain beneficial biological results associated with caloric restriction comprising administration of a composition containing at least one active agent which blocks use of glucose as a source of energy in cells in amounts sufficient to lower tissue glucose level and decrease in plasma insulin levels in the non-diabetic animal.

### DESCRIPTION OF THE INVENTION

It is the purpose of this invention to provide benefits associated with caloric restriction by controlled administration of antimetabolites of glucose. Judicious use of compounds that block the normal metabolism of cellular glucose can result in changes in physiological function that are similar to those arising from caloric restriction. The compounds and compositions used in accord with the teachings herein often lower body temperature. Such lowering of body temperature and slowing of the rate of metabolism in the tissues often is beneficial in treatment of trauma and in other treatment modalities where decrease in metabolic rate is desirable.

Two related aspects must be addressed. Glucose is used by cells both as an energy source (catabolic mode) and for incorporation into other compounds (anabolic mode). Inhibition or interference with anabolic uses of glucose should be avoided, since this may lead to production of anomalous glycoproteins and glycolipids and eventually to undesired side effects. It should be noted that various non-nutritious sweet compounds (some of them carbohydrates) have been suggested as agents to reduce obesity based on the theory that, if these compounds can not be a source of energy, caloric intake may be reduced. The instant invention does not relate simply to agents that lack nutritional value. These prior art agents that have been used simply to avoid/treat obesity perform a different function and do not provide the benefits sought in the practice of the instant invention.

Decreased Utilization of Glucose as Energy Source by 2-deoxy-D-glucose:

To fully mimic the beneficial effects of caloric restriction, it is necessary that glucose anti-metabolites be given over an extended time period. Previous studies clearly show that it is not possible to administer compounds such as 2-deoxy-D-glucose in high doses, since significant untoward side effects and toxicity have often been observed. However, studies in rodents (Lane et al., J. Anti-Aging Med. 1 (4):327-337, 1998) have shown that long-term disruption of glucose metabolism using a lower dose of 2 deoxy-D-glucose can mimic some of the major metabolic hallmarks of caloric restriction, including reduced body temperature, weight loss, and lower fasting insulin levels.

In light of the above potential physiologic benefits of caloric restriction weighed against the negative aspects of metabolic inhibition by 2-deoxy-D-glucose, alternatives which act as antimetabolites of glucose without the potentially harmful side effects are preferred for purposes of practicing the invention.

Decrease of Availability of Glucose to Cells by 5-thio-D-glucose

5-Thioglucose, an analog of glucose, has (in vivo) more pronounced effects than 2-deoxy-D-glucose. The compound is believed to act mainly by inhibiting glucose uptake by cells. The majority of 5-thioglucose (97%) injected into a rat has been found excreted unchanged in urine (Hoffman et al., Biochemistry 7, pp 4479-4483 (1968)). 5-Thioglucose is remarkably non-toxic; LD₅₀ was measured to be 14 g/kg, by injection, in rats (Chen et al., Arch. Biochem. Biophys., 169, pp 392-396 (1975)).

Since 5-thioglucose seems to be excreted unchanged in urine, this compound presents certain advantages for chronic administration over 2-deoxy-D-glucose. Nevertheless, since 5-thioglucose inhibits glucose uptake, appropriate dosing can result in benefits associated with caloric restriction.

### Effects of 3-O-methylglucose

This analog of glucose, in contrast with 2-deoxy-D-glucose, is not metabolized (Jay et al., J. Neurochem. 55, pp. 989-1000 (1990)) and, thus may provide certain advantages for use in chronic administration. In the context of this invention, 3-O-methylglucose can prevent utilization of glucose as an energy source as demonstrated by response to its administration in rats. The responses were about seven times weaker than those to 2-deoxyglucose.

Effects of Anhydrosugars: 1,5-anhydro-D-glucitol (polygalitrol):

This compound is a non-reducing analog of glucose and is enzymatically converted to 1,5-anhydroglucitol-6-phosphate, albeit the conversion is less efficient than that of 2-deoxyglucose (Sols et al., J. Biol. Chem., 210, pp 581-595 (1954)). 1,5-anhydroglucitol-6-phosphate is an allosteric (non-competitive) inhibitor of hexokinase, which catalyzes the first and the regulatory step of the entire glycolysis (Crane et al., J. Biol. Chem., 210, pp. 597-696 (1954)). Furthermore 1,5 anhydroglucitol-6-phosphate is a non-reducing analog and cannot be a substrate for the next step of glycolysis catalyzed by glucose 6-phosphate isomerase. Consequently, this analog could accumulate in cells and act as a very effective metabolic block to glucose utilization. Another advantage relating to its non-reducing character is that this compound cannot be incorporated into glycolipids, glycoproteins and glycogen. Thus, its effects are specific to glycolysis and would not be expected to affect other metabolic processes or exert toxicity of some glucose anti-metabolites previously discussed.

Interestingly, this compound (or its phosphate) has been found in the human body. It would found to be present in cerebrospinal fluid of patients who had occasional high blood glucose (from diabetes and diseases of kidney) in large enough concentrations to be detected in tests performed in normal clinical settings.

Use of 2,5-anhydro-D-mannitol and 2,5-anhydroglucitol:

These compounds are non-reducing analogs of fructose. Fructose is an important component of food and fructose phosphates and diphosphate are intermediate products of glycolysis. Nevertheless, inhibition of metabolic events involving fructose and its phosphates by anhydrosugar analogs is difficult. Alpha and beta anomers of fructose, which spontaneously inter-convert, correspond to different anhydrosugars, to 2,5-anhydroglucitol and 2,5-anhydromannitol, respectively. Thus, only a few of the enzymatic conversions can be inhibited by a single compound. The 2,5-anhydromannitol has been investigated in some detail. That compound is taken up by cells and converted into 2,5-anhydromannitol-1-phosphate. That phosphate is an analog of fructose-1-phosphate, but can not be cleaved by the aldolase and, therefore, the utilization of both glucose and fructose by cells is blocked. The 2,5,-anhydromannitol had been found to interfere in glucose formation and utilization in isolated rat hepatocytes (Riquelme et al., Proc. Natl. Acad. Sci. USA, 80, pp 431-435 (1983)).

### Decrease of Glucose Utilization as Energy Source by Ketoses.

Mannoheptulose is present in reasonable amounts in some foods (e.g. some avocados contain up to 5% of the wet weight) and can be classified as a “generally recognized as safe” substance for the human consumption. In studies of metabolism, 10 grams of mannoheptulose have been safely administered to humans orally. About 5% of the mannoheptulose ingested was reported to appear in urine after oral dosing. The fate of injected mannoheptulose has previously been investigated in rats: 66% was excreted unchanged, 29% was metabolized, and, a day after the injection, 5% remained in the body (Simon et al., Arch. Biochem. Biophys., 69, pp. 592-601 (1957)).

### EXAMPLE I

### Preparation of Mannoheptulose-Containing Supplement

Fresh avocadoes (Lula variety) were obtained from Fresh King Incorporated (Homestead, Fla.). The avocados were manually split open and the pits were removed and discarded. The remaining skin and pulp were ground through a Hobart Commercial Food Preparation machine (serial # 11-10410235) using a 12¼ sieve. The ground avocado was then transferred to an Edwards Freeze Drier (Super Modulyo Model, Crawely, Sussex, England). The freeze drier was set at −20° C. for the first 24 hours, −5° C. for the following 24 hours and 5° C. for the final 72 hours. Upon removal from the freeze drier, the meal was ground to a powder using a Straub Grinding Mill (model 4E, Philadelphia, Pa.). The avocado meal was analyzed and found to contain 10.35% mannoheptulose. (It should be noted that the amount of mannoheptulose found in avocados various with the particular strain, some avocados having little or no mannoheptulose.)

### EXAMPLE II

### Administration of Mannoheptulose to Beagle Dogs

The use of mannoheptulose for purposes of obtaining benefits associated with inhibiting metabolism of glucose was tested in beagle dogs. A total of 12 beagles were utilized for the study and were fed a standard commercial diet throughout the study period. Fasting blood samples were drawn 7, 6, 4, and 2 days prior to administration of mannoheptulose. The mannoheptulose was delivered to the dogs in the form of a freeze-dried avocado meal containing 10 to 12% mannoheptulose. This preparation was adjusted to provide mannoheptulose doses of 2, 20 and 200 mg/kg body weight (MH-2, MH-20, MH-200, respectively). Fasting blood samples were collected 1, 3, 5 and 7 days after initiation of the administration of mannoheptulose.

### RESULTS

Insulin levels were lowered by up to 35% in dogs who had received the avocado meal when compared to those dogs on similar diets who had not received meal with their diets. Those changes were similar to the decreases found in mammals on caloric restricted diets. In contrast, plasma glucose concentrations of dogs fed the same standard diet which did not contain the avocado meal did not show such effects.

The mechanism by which insulin is reduced relates to the fact that glucose must be metabolized by the pancreas to stimulate insulin secretion (German et al., Proc. Nat. Acad. Sci. 90:1781-1785. 1993). Mannoheptulose is thought to inhibit glucokinase, the initial enzyme involved in glucose metabolism in pancreas and liver. Therefore, reduced insulin levels indicate that mannoheptulose has indeed inhibited glucose metabolism. This effect on glucokinase by mannoheptulose would indicate use of mannoheptulose directed at inhibition of tumor growth as an alternative to administration of 2-deoxy-D-glucose. (See Board, M., et al., Cancer Res. 55(15): 3278-3285. 1995.) Mannoheptulose would present a safe alternative to 2-deoxy-D-glucose, since it would avoid some untoward effects seen when 2-deoxy-D-glucose is administered on a long-term basis.

The availability of glucose to cells can also be decreased using other dietary supplements than those specifically identified herein which have similar effect on metabolism of glucose that can result in an inhibition of glucose processing.

The methods of the invention may be practiced by administering the active agents orally or parenterally, though oral administration would be the norm. When lowering of tissue metabolism is desired, as an adjunct to treatment of trauma, the active agents may be administered intravenously.

Dosage will depend on the agent used and will vary depending on the extent of lowering of tissue metabolism that is desired and the size and condition of the animal to which the agent is to be administered. Dosage in the range of 0.001 g/kg to about 1 g/kg would be suggested. Dosage at the lower range would be appropriate when using 2-deoxy-D-glucose in large mammals. Higher dosage, particularly of compounds such as 5-thio-D-glucose or mannitol should be readily tolerated.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to the term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A composition comprising mannoheptulose wherein said composition is adapted for use by a dog.

2. The composition of claim 1 wherein said mannoheptulose is obtained from an avocado.

3. The composition of claim 1 comprising from about 10% to about 12% mannoheptulose.

4. A composition comprising mannoheptulose wherein said composition lowers plasma insulin levels and is adapted for use by a dog.

5. The composition of claim 4 wherein said mannoheptulose is obtained from an avocado.

6. The composition of claim 4 comprising from about 10% to about 12% mannoheptulose.

7. A composition for a dog, said composition comprising mannoheptulose.

8. The composition of claim 7 comprising from about 10% to about 12% mannoheptulose.

9. The composition of claim 7 wherein said mannoheptulose is obtained from an avocado.

10. A method of lowering plasma insulin levels in a dog, said method comprising the step of administering a composition comprising mannoheptulose to said dog.

11. The method of claim 10 wherein administration is selected from the group consisting of orallay, parenterally, and combinations thereof.

12. The method of claim 11 wherein administration is orally.

13. The method of claim 10 wherein said mannoheptulose is obtained from an avocado.

14. The method of claim 10 wherein said composition comprises from about 10% to about 12% mannoheptulose.
